(19) United States
(12) Patent Application Publication
(10) Pub. No.: US 2005/0238598 A1
(43) Pub. Date: Oct. 27, 2005

(54) Antiperspirant compositions and method comprising wax microparticles

(76) Inventors: Lionnel Aubert, Asnieres Sur Oise (FR); Veronique Douin, Saint-Nom la Breteche (FR); Roxane Provost, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/100,519

(22) Filed: Apr. 7, 2005

Related U.S. Application Data
(60) Provisional application No. 60/587,042, filed on Jul. 13, 2004.

(30) Foreign Application Priority Data
Apr. 7, 2004 (FR) 04 50698

(51) Int. Cl. A61K 7/32; A61K 7/36; A61K 7/035

(52) U.S. Cl. 424/67; 424/69

(57) ABSTRACT

The present disclosure relates to a cosmetic composition comprising, in an oil-in-water emulsion: (A) at least one antiperspirant active agent; and (B) a plurality of wax particles having a mean particle size of less than 50 μm and a melting point of greater than 80° C. The present disclosure further relates to a cosmetic process for treating perspiration and body odors related to perspiration comprising applying, to the surface of skin, an effective amount of such a composition. The present disclosure also relates to a method for reducing the drying time and the stickiness effect of an antiperspirant composition comprising adding a plurality of wax particles having a mean size of less than 50 μm and a melting point of greater than 80° C. in the antiperspirant composition.

This application claims benefit of U.S. Provisional Application No. 60/587,042, filed Jul. 13, 2004, and of French Application No. 04/50698, filed Apr. 7, 2004, both of which are incorporated herein by reference.

The present disclosure relates to a cosmetic composition comprising, in an oil-in-water emulsion:
(A) at least one antiperspirant active agent; and
(B) a plurality of wax particles having a mean size of less than 50 μm and a melting point of greater than 80° C.

The present disclosure also relates to a cosmetic process for the treatment of body odors related to perspiration (e.g., auxiliary odors, odors of the feet) comprising applying, to the surface of skin, an effective amount of such a composition.

In the cosmetic field, it is well known to use, in topical application, deodorant products comprising active substances of antiperspirant type, of bactericide type, or of the odor-absorbing type to reduce and even eliminate generally unpleasant body odors.

Antiperspirant substances have the effect of limiting the flow of sweat. They are generally composed of aluminum salts.

Many different types of antiperspirant compositions have been described in the literature and have appeared on the market in forms such as gels, sticks, creams, roll-ons, or aerosols.

Three main types of formulations exist among the roll-ons or creams commercially available in the field of antiperspirant products: (1) anhydrous or soft solid formulations, (2) water-in-oil emulsions, and (3) oil-in-water emulsions.

Anhydrous or soft solid formulations and water-in-oil emulsions, which are vehicles comprising a continuous oily phase, exhibit the disadvantage of producing a greasy feel on application, a lack of feeling of freshness, and even an inadequate deodorant effectiveness.

Antiperspirant oil-in-water emulsions comprising a continuous aqueous phase are, for example, desired for their novel cosmetic qualities with regard to their penetration, their spreading, and the feeling of freshness on the skin after application. The compositions currently on the market exhibit, as disadvantages:
a) a wet effect upon application, which may result in transfer of the product on contact with the clothes and which is due to excessively slow drying; and/or
b) a stickiness effect due to the presence of antiperspirant salts, which are in the dissolved state in the aqueous phase.

Thus, it would be desirable to provide an antiperspirant oil-in-water emulsion, the drying time of which is shorter, and the stickiness effect of which, due to the presence of antiperspirant salts dissolved in the aqueous phase, is substantially reduced and even eliminated.

A description of the use of micronized polyethylene waxes as suspending agents for aluminum salts in antiperspirant formulations of the roll-on type is found in the paper by Harvey M. Fishmann in the review Happi August 1998, page 40. Micronized polyethylene waxes of the Microthene® or Acumist type are also known in International Patent Application Publication Nos. WO 01/85119 and WO 03/105795 as fillers in antiperspirant formulations in the form of water-in-oil emulsions. In U.S. Pat. Nos. 5,294,447 and 5,178,881, and European Patent Application No. EP 981 324, polyethylene waxes of the Microthene® type are used as drying agents in anhydrous antiperspirant formulations, in which the antiperspirant active agent, i.e., aluminum salt, is in suspension in the continuous oily phase.

The present inventors have discovered that by adding wax particles having a mean particle size of less than 50 μm and a melting point of greater than 80° C. to oil-in-water emulsions based on antiperspirant active agents, rapid drying times, a greatly reduced stickiness effect, and/or a good deodorant effectiveness compared with oil/water emulsions of the prior art may be obtained.

In one embodiment, the present disclosure is thus a cosmetic composition comprising, in an oil-in-water emulsion:
(A) at least one antiperspirant active agent; and
(B) a plurality of wax particles having a mean size of less than 50 μm and a melting point of greater than 80° C.

Another aspect of the present disclosure is using the composition as defined above to reduce the flow of sweat and/or to mask, improve or reduce the unpleasant odor resulting from the decomposition of human sweat by bacteria.

A further aspect of the present disclosure is a process for the treatment of perspiration comprising applying, to the surface of the skin, an effective amount of such a composition.

### BRIEF DESCRIPTION OF THE DRAWING

FIG. 1 is a comparison of the variation in the strength of the stickiness as a function of the time of Examples 1 and 2.

### DESCRIPTION

The treatment of perspiration may comprise both reducing and/or suppressing the unpleasant odor brought about by sweat and reducing and/or suppressing the feeling of dampness produced by sweat.

In one embodiment, a process for treating body odors related to perspiration (e.g., auxiliary odors and/or odors of the feet) comprises applying, to the surface of the skin, an effective amount of such a composition.

According to one embodiment, a method of using a plurality of wax particles having a mean size of less than 50 μm and a melting point of greater than 80° C. in an antiperspirant composition of the oil-in-water emulsion type reduces the drying time and the stickiness effect after application to the skin.

As used herein, the term “antiperspirant active agent” means any substance capable of reducing the flow of sweat.

The antiperspirant active agents which may be used according to the disclosure are, for example, chosen from aluminum and zirconium salts; and complexes of zirconium chlorohydrate and of aluminum chlorohydrate with an amino acid.

Mention may be made, for example, of aluminum salts, aluminum chlorohydrate in the activated or nonactivated form, aluminum chlorohydrex, aluminum. chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichlorohydrate, aluminum sesquichlorohydrex polyethylene glycol complex, aluminum sesquichlorohydrex propylene glycol complex, or aluminum sulphate buffered by sodium aluminum lactate.

Mention may be made, for example, of aluminum and zirconium salts, aluminum zirconium octachlorohydrate, aluminum zirconium pentachlorohydrate, aluminum zirconium tetrachlorohydrate, or aluminum zirconium trichlorohydrate.

The complexes of zirconium chlorohydrate and of aluminum chlorohydrate with an amino acid are generally known under the name ZAG (when the amino acid is glycine). They are disclosed, for example, in U.S. Pat. No. 3,792,068. The ZAG complexes usually exhibit an Al/Zr quotient ranging from about 1.67 to 12.5 and a Metal/Cl quotient ranging from about 0.73 to 1.93. Mention may be made, among these products, to aluminum zirconium octachlorohydrex glycine, aluminum zirconium pentachlorohydrex glycine, aluminum zirconium tetrachlorohydrex glycine, and aluminum zirconium trichlorohydrex glycine complexes.

According to one embodiment, the antiperspirant active agents may be chosen from aluminum chlorohydrate in activated or nonactivated form, such as the product sold by Reheis under the name Microdry Aluminum Chlorohydrate® or by Guilini Chemie under the name Aloxicoll PF 40®. Use may also be made of the commercial product Reach 103® manufactured by Reheis or the commercial product Westchlor 200 manufactured by Westwood.

The antiperspirant active agents are present in the composition according to the present disclosure in an amount ranging from about 0.001% to 40% by weight with respect to the total weight of the composition and for example, from 0.1% to 25% by weight.

The plurality of micronized wax particles in accordance with the present disclosure, for example, have a mean particle size of less than 20 μm and further for example, from 1 μm to 15 μm. They exhibit a melting point such as greater than 100° C. Their density at 25° C. generally varies ranging from 0.9 g/cm³ to 2.5 g/cm³.

The plurality of micronized wax particles in accordance with the present disclosure may be composed of wax chosen from natural waxes of mineral, fossil, animal and vegetable origin; synthetic waxes, and their mixtures.

Mention may be made, among natural mineral waxes, of microcrystalline waxes, paraffin wax, petrolatum wax, paraffin waxes, ozokerite, or montan wax.

Mention maybe made, among natural animal waxes, of beeswax or lanolin, and its derivatives.

Mention may be made, among natural vegetable waxes, of candelilla waxes, ouricury waxes, carnauba waxes, Japan wax or cocoa butter or cork fibre or sugarcane waxes; hydrogenated oils which are solid at 25° C., such as hydrogenated jojoba oil, hydrogenated castor oil, hydrogenated palm oil or hydrogenated coconut oil; fatty esters; and glycerides.

Mention may be made, among synthetic waxes, of silicone waxes, waxes obtained by Fischer-Tropsch synthesis, polyethylene waxes, polytetrafluoroethylene (PTFE) waxes or their mixtures. Mention may also be made of the mixtures of waxes disclosed in the International Patent Application Publication No. WO 00/3104330.

The plurality of micronized wax particles according to the present disclosure may be composed of a mixture of at least one natural wax and at least one synthetic wax. The plurality of micronized wax particles according to the disclosure may also be composed of a mixture of several natural waxes or of a mixture of several synthetic waxes.

Mention may be made, among the micronized natural wax particles which may be used according to the present disclosure, of carnauba wax particles with a melting point ranging from 83° C. to 86° C., a density of 0.99 g/cm³, and a mean size ranging from 6 μm to 8 μm, such as the commercial product sold under the trade name Microcare 350® by Micro Powders.

Mention may be made, among the plurality of micronized wax particles comprised of a mixture of natural wax and of synthetic wax which may be used according to the present disclosure, of particles composed of carnauba wax and of polyethylene wax having a melting point ranging from 107° C. to 114° C., a density ranging from 0.96 g/cm³ to 0.97 g/cm³, and a mean particle size ranging from 4 μm to 6 μm, such as the commercial products sold under the commercial names Microcare 300® and Microcare 310® by Micro Powders.

Mention may be made, among the plurality of micronized particles based on synthetic wax which may be used according to the present disclosure, of:
polytetrafluoroethylene particles which have a melting point of greater than 316° C., a density of approximately 2.2, and a mean particle size ranging from 5 μm to 13 μm, such as the products sold under the trade names Microslip 519 and Microslip 519 L by Micro Powders,
particles comprised of a mixture of polyethylene wax and of polytetrafluoroethylene wax which have a melting point ranging from 115° C. to 125° C., a density of 0.99 g/cm³ to 1.1 g/cm³, and a mean particle size ranging from 9 to 12, such as the products sold under the trade names Microsilk 418® and Microsilk 419® by Micro Powders,
polyethylene particles which have a melting point of greater than 100° C., a density of 0.96 g/cm³ to 0.98 g/cm³, and a mean particle size ranging from 5 μm to 40 μm, such as the products sold under the trade names Microthene FN-500® and Microthene FN-510® by Equistar Chemical LP or the products sold under the trade names Acumist A-6® and Acumist B-18® by Allied Signal Corp.

According to one embodiment of the present disclosure, use will be made of polyethylene particles with a mean size ranging from 1 μm to 12 μm, a melting point ranging from 105° C. to 135° C., and a density ranging from 0.94 g/cm³ to 0.97 g/cm³, such as the polyethylene powders sold under the trade names Micropoly 200®, 210®, 220®, 220L® and 250 S® by Micro Powders.

The plurality of wax particles according to the present disclosure are, for example, present in an amount ranging from 0.5% to 15% by, weight and further for example, from 1% to 10% by weight, with respect to the total weight of the composition.

The compositions according to the present disclosure may further comprise at least one additional deodorant active agent.

As used herein, the term “deodorant active agent” means any substance capable of reducing and/or suppressing the flow of sweat and/or of absorbing human sweat and/or of masking, absorbing, improving or reducing the unpleasant odor resulting from the decomposition of human sweat by bacteria.

The at least one additional deodorant active agent may be bacteriostatic agents or bactericidal agents, such as 2,4,4′-trichloro-2′-hydroxydiphenyl ether (Triclosan®), 2,4-dichloro-2′-hydroxydiphenyl ether, 3′,4′,5′-trichlorosalicylanilide, 1-(3′,4′-dichlorophenyl)-3-(4′-chlorophenyl) urea (Triclocarban®) or 3,7,11-trimethyldodeca-2,5,10-trienol (Farnesol®); or quaternary ammonium salts, such as cetyltrimethylammonium salts or cetylpyridinium salts.

Mention may also be made, among the at least one additional deodorant active agent, of zinc salts, such as zinc salicylate, zinc sulphate, zinc chloride, zinc lactate or zinc phenylsulphonate; chlorhexidine and its salts; sodium bicarbonate; diglycerol monocaprate, diglycerol monolaurate or glycerol monolaurate; or polyhexamethylene biguanide salts.

The at least one additional deodorant active agent may be present in the composition according to the present disclosure in an amount ranging from about 0.001% to 40% by weight, with respect to the total weight of the composition, and further for example, from 0.1% to 25% by weight.

The antiperspirant cosmetic composition according to the present disclosure may be provided in a more or less thickened form distributed in a tube or in a twist stick or in the form of a roll-on packaged in the ball form and can, in this respect, comprise the ingredients generally used in products of this type and well known to a person skilled in the art.

The antiperspirant cosmetic composition according to the present disclosure, which is an oil-in-water emulsion, generally comprises at least one emulsifier.

Mention may be made, as emulsifiers, for example, of nonionic emulsifiers, such as oxyalkylenated (e.g., polyoxyethylenated) esters of fatty acids and of glycerol; oxyalkylenated esters of fatty acids and of sorbitan; oxyalkylenated (oxyethylenated and/or oxypropylenated) fatty acid esters; oxyalkylenated (oxyethylenated and/or oxypropylenated) fatty alcohol ethers; and esters of sugars, such as sucrose stearate and their mixtures, such as the mixture of glyceryl stearate and of PEG-40 stearate.

Mention may also be made of, for example, fatty alcohol/alkylpolyglycoside emulsifying mixtures, as are disclosed in International Patent Application Publication Nos. WO 92/06778, WO 95/13863, and WO 98/47610, for example the commercial products sold by SEPPIC under the Montanov® names.

The fatty phase in accordance with the oil-in-water emulsions according to the disclosure comprises at least one hydrophobic compound, which renders the phase immiscible in water.

The fatty phase, for example, further comprises at least one volatile or nonvolatile emollient oil of silicone or hydrocarbon nature. These emollient oils are disclosed, for example, in U.S. Pat. Nos. 4,822,596 and 4,904,463.

The at least one emollient oil according to the present disclosure may be chosen from products of the volatile silicone type, nonvolatile silicones, and other nonvolatile emollients.

As used herein, the term “volatile silicones” is defined in a known way as compounds, which are volatile at ambient temperature (20° C.-25° C.). They generally exhibit a vapor pressure at 25° C. ranging from 1 Pa or 10 Pa to 2 kPa.

Mention may be made, among these compounds, of cyclic and linear volatile silicones of the dimethylsiloxane type, the chains of which comprise from 3 to 9 silicone residues. For example, the tetramer (D4), pentamer (D5) and hexamer (D6) cyclomethicones may be chosen.

As used herein, the term “nonvolatile silicones” is defined in a known way as compounds with a low vapor pressure at ambient temperature. The following are included among these compounds: polyalkylsiloxanes, for example linear polyalkylsiloxanes, such as the linear polydimethylsiloxanes or dimethicones sold by Dow Coming under the name of “Dow Corning 245 Fluid®”; polyalkylarylsiloxanes, such as, for example, the polymethylphenylsiloxanes sold by Dow Corning under the name of “Dow Corning 556 Fluid®”; or polyether and siloxane copolymers, such as, for example, dimethicone copolyols.

Mention may be made, among the nonvolatile emollient oils of hydrocarbon type which may be used in the present disclosure, of, for example: liquid hydrocarbon derivatives, such as mineral oils and hydrogenated polyisobutenes and for example, polydecenes, paraffins and isoparaffins having at least 10 carbon atoms; fatty alcohols which are liquid at ambient temperature, such as isostearyl alcohol or octyldodecanol; aliphatic esters of C₃-C₁₈ alcohols with C₃-C₁₈ acids, such as isopropyl myristate, lauryl myristate, isopropyl palmitate, diisopropyl sebacate or diisopropyl adipate; aromatic esters of benzoic acid with C₁₂-C₁₈ alcohols and their mixtures, such as C₈-C₁₈ alkyl benzoates; ethers of aliphatic fatty alcohols, such as ethers of myristyl alcohol, for example PPG-3 myristyl ether; and (C₁-C₄)alkyl ethers of polyglycols, such as PPG4 butyl ether.

In order to improve the homogeneity of the product, use may be made of at least one suspending agent, which is, for example, chosen from hydrophobic modified montmorillonite clays, such as hydrophobic modified bentonites and hectorites. Mention may be made, for example, of the product Stearalkonium Bentonite (CTFA name) (i.e., reaction product of bentonite and of the quaternary ammonium stearalkonium chloride), such as the commercial product sold under the name Tixogel MP 250® by Sud Chemie Rheologicals, United Catalysts Inc., or the product Disteardimonium Hectorite (CTFA name) (i.e., reaction product of hectorite and of distearyldimonium chloride), sold under the name of Bentone 38 or Bentone Gel by Elementis Specialities.

The at least one suspending agent is, for example, present in an amount ranging from 0.1% to 5% by weight and further for example, from 0.2% to 2% by weight, with respect to the total weight of the composition.

The compositions according to the present disclosure may further comprise at least one organic powder.

Mention may be made, among the fillers which may be used according to the present disclosure, of organic powders. As used herein, the term “organic powder” is understood to mean any solid, which is insoluble in the medium at ambient temperature (25° C.).

Mention may be made, as organic powders which may be used in the composition of the present disclosure, for example, of polyamide particles and for example, those sold under the names Orgasol® by Atochem; microspheres based on acrylic copolymers, such as those made of ethylene glycol dimethacrylate/lauryl methacrylate copolymer sold by Dow Coming under the name Polytrap®; poly(methyl methacrylate) microspheres sold under the name Microsphere M-100® by Matsumoto or under the name Covabead LH85® by Wackherr; powders formed of ethylene-acrylate copolymer, such as those sold under the name Flobeads® by Sumitomo Seika Chemicals; expanded powders, such as hollow microspheres and for example, the microspheres formed from a terpolymer of vinylidene chloride, of acrylonitrile and of methacrylate sold under the name Expancel® by Kemanord Plast under the references 551 DE 12® (particle size of approximately 12 μm and density 40 kg/m³), 551 DE 20® (particle size of approximately 30 μm and density 65 kg/m³) or 551 DE 50® (particle size of approximately 40 μm), or the microspheres sold under the name Micropearl F 80 ED® by Matsumoto; powders formed from natural organic materials, such as powders formed from starch, such as from crosslinked or noncrosslinked maize, wheat or rice starches, such as the powders formed from starch crosslinked by octenylsuccinic anhydride sold under the name Dry-Flo by National Starch; silicone resin microbeads, such as those sold under the name Tospearl® by Toshiba Silicone, such as Tospearl 240; or powders formed from amino acids, such as the lauroyllysine powder sold under the name Amihope LL-11® by Ajinomoto.

The aqueous phase of the emulsions may comprise water and/or other water-soluble or water-miscible solvents. The water-soluble or water-miscible solvents comprise short-chain, for example C₁-C₄, monoalcohols, such as ethanol or isopropanol; or diols or polyols, such as ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether and sorbitol. For example, the water-soluble or water-miscible solvents are propylene glycol and glycerol.

The cosmetic composition according to the present disclosure may further comprise at least one cosmetic adjuvant chosen from softeners, antioxidants, opacifiers, stabilizers, moisturizing agents, vitamins, fragrances, bactericides, preservatives, polymers, thickening agents, and any other ingredient commonly used in cosmetics for this type of application.

Of course, a person skilled in the art will take care to choose this or these optional additional compounds so that the advantageous properties intrinsically attached to the cosmetic composition in accordance with the present disclosure are not, or not substantially, detrimentally affected by the envisaged addition or additions.

The thickeners, such as nonionic thickeners, may be chosen from modified and unmodified guar gums and celluloses, such as hydroxypropylated guar gum, cetylhydroxyethylcellulose and silicas, such as, for example, Bentone Gel Mio®, sold by NL Industries, or Veegum Ultra®, sold by Polyplastic.

The amounts of these various constituents which may be present in the cosmetic composition according to the present disclosure are those conventionally used in antiperspirant roll-ons, twist sticks, and creams.

The compositions according to the present disclosure may also comprise at least one additional structuring or gelling agent for the oily phase, such as those mentioned above in the description, such as waxes and/or linear solid fatty alcohols; fatty acids or their salts (e.g., stearic acid, sodium stearate, 12-hydroxystearic acid); dibenzylidene alditols (DBS); Ianosterol; N-acylamino acid derivatives; derivatives of di- or tricarboxylic acids, such as alkyl-N,N′-dialkylsuccinamides (i.e.: dodecyl-N,N′-dibutylsuccinamide); or organopolysiloxane elastomers, such as those disclosed in International Patent Application Publication No. WO 97/44010.

The composition according to the present disclosure may also be pressurized and may be packaged in an aerosol; it then comprises at least one propellant generally used in products of this type which are well known to a person skilled in the art, such as, for example, dimethyl ether; or volatile hydrocarbons, such as n-butane, propane, isobutane and their mixtures, optionally with at least one chlorinated and/or fluorinated hydrocarbon; mention may be made, among the latter, of the compounds sold by Dupont de Nemours under the names Freon® and Dymel® and for example, monofluorotrichloromethane, difluorodichloromethane, tetrafluorodichloroethane, and 1,1-difluoroethane, sold for example, under the trade name Dymel 152 A® by Dupont.

Use may also be made, as the at least one propellant, of carbon dioxide gas, nitrous oxide, nitrogen, or compressed air.

The at least one propellant may be present in an amount ranging from 20% to 85% by weight of the total weight of the composition.

In one embodiment of the present disclosure, a cosmetic process for treating human perspiration comprises applying, to an auxiliary surface, i.e., skin, an effective amount of a composition as defined above is provided.

According to one embodiment of the present disclosure, a process for treating human body odors comprises applying, to an auxiliary surface, an effective amount of a composition as defined above.

According to another embodiment of the present disclosure, a method for using a plurality of wax particles having a mean size of less than 50 μm and a melting point of greater than 80° C. in an antiperspirant composition of the oil-in-water emulsion type reduces the drying time and the stickiness effect of the composition after application to the skin.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements.

The present disclosure may be understood more clearly with the aid of non-limiting examples that follow.

### EXAMPLES

The two antiperspirant roll-ons with the following compositions were prepared:

| Phase | Ingredients | Ex. 1 | Ex. 2 (*) |
|---|---|---|---|
| A | PPG-15 stearyl ether | 3.00 | 3.00 |
| | (Arlamol E ®) | | |
| | Dimethicone 200 cst | 0.50 g | 0.50 g |
| | (Dow Corning 200 Fluid ®) | | |
| | Ceteareth-33 | 1.25 | 1.25 |
| | (Simulsol CS ®) | | |
| | Cetearyl alcohol | 2.50 | 2.50 |
| C | Polyethylene wax particles with a | 5 | NA |
| | mean size of 8-10 μm, a melting | | |
| | point of 123-125° C. and a density | | |
| | of 0.96 (Micropoly 220 L ® from | | |
| | Micro Powders) | | |
| B | Aluminium chlorohydrate as a 50% | 30.0 g | 30.0 g |
| | aqueous dispersion (Chlorhydrol ® | | |
| | 50% USP) | | |
| | Water q.s. for | 100 g | 100 g |
| D | Fragrance | 1.0 | 1.00 |
| | Preservative | q.s. | q.s. |

(*): outside the present disclosure
q.s. = quantity sufficient

The roll-ons of Examples 1 and 2 were prepared according to the following procedure:

Phases A and B were each brought to a temperature of about 80° C.

The emulsion was prepared with stirring by pouring A into B.

The emulsion was cooled to approximately 40° C. and D and then C were introduced with stirring.

A comparative test on the drying time and on the stickiness effect of each roll-on after application was subsequently carried out according to the following protocol:

A texture analyzer was used with the following parameters:

| | |
|---|---|
| Equipment | Texture Expert TA-XT2 ® by Rheo |

Presettings

| | |
|---|---|
| Rotor: | 10 mm ebonite cylinder |
| Descent prerate of the rotor | 2 mm/s |
| Descent rate of the rotor | 10 mm/s |
| Ascent rate of the rotor | 5 mm/s |
| Force exerted on the glass plate: | 40 g |
| Displacement: | 0.5 mm |
| Time | 1.5 s |
| Activating force | 20 g |
| Acquisition rate | 200 pps |

Each antiperspirant of Example 1 and 2 was applied with a film draw to a glass plate. The maximum adhesive force exerted by the rotor, which corresponds to the strength of the stickiness, was measured every minute and the rotor was cleaned after each measurement.

This operation was carried out until the maximum force is zero, namely until the completion of the drying. The drying time was determined at this precise moment.

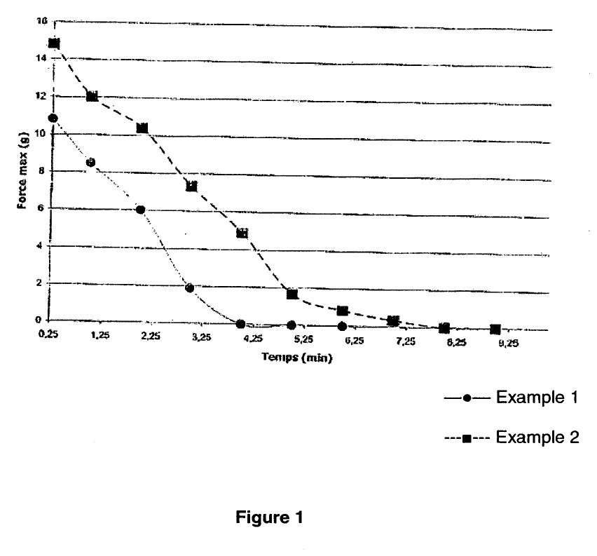

The results of the tests are represented in FIG. 1 by the curve of variation in the strength of the stickiness as a function of the time.

It was found that the antiperspirant roll-on of Example 1 according to the disclosure comprising micronized polyethylene particles dried much more rapidly and exhibited a substantially weaker stickiness effect than the filler-free antiperspirant roll-on of Example 2.

1. A cosmetic composition comprising, in an oil-in-water emulsion:
(A) at least one antiperspirant active agent; and
(B) a plurality of wax particles with a mean size of less than 50 μm and a melting point of greater than 80° C.

2. The composition according to claim 1, wherein the at least one antiperspirant active agent is chosen from aluminum and zirconium salts; and complexes of zirconium chlorohydrate and of aluminum chlorohydrate with an amino acid.

3. The composition according to claim 2, where the at least one antiperspirant active agent is chosen from aluminum chlorohydrate in the activated and nonactivated form, aluminum chlorohydrex, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichlorohydrate, aluminum sesquichlorohydrex polyethylene glycol complex, aluminum sesquichlorohydrex propylene glycol complex, and aluminum sulphate buffered by sodium aluminum lactate.

4. The composition according to claim 2, wherein the at least one antiperspirant active agent is chosen from aluminum zirconium octachlorohydrate, aluminum zirconium pentachlorohydrate, aluminum zirconium tetrachlorohydrate, and aluminum zirconium trichlorohydrate.

5. The composition according to claim 2, wherein the at least one antiperspirant active agent is chosen from complexes of zirconium chlorohydrate and of aluminum chlorohydrate with glycine (ZAG).

6. The composition according to claim 5, wherein the at least one antiperspirant active agent is chosen from aluminum zirconium octachlorohydrex glycine, aluminum zirconium pentachlorohydrex glycine, aluminum zirconium tetrachlorohydrex glycine, and aluminum zirconium trichlorohydrex glycine complexes.

7. The composition according to claim 2, wherein the at least one antiperspirant active agent is aluminum chlorohydrate in the activated or nonactivated form.

8. The composition according to claim 1, wherein the at least one antiperspirant active agent is present in an amount ranging from 0.001% to 40% by weight, relative to the total weight of the composition.

9. The composition according to claim 8, wherein the at least one antiperspirant active agent is present in an amount ranging from 0.1% to 25% by weight, relative to the total weight of the composition.

10. The composition according to claim 1, wherein the wax particles have a mean particle size of less than 20 μm, a melting point of greater than 100° C., and a density at 25° C. ranging from 0.9 g/cm³ to 2.5 g/cm³.

11. The composition according to claim 1, wherein the wax particles are comprised of wax chosen from natural waxes of mineral, fossil, animal and vegetable origin; synthetic waxes; and mixtures thereof.

12. The composition according to claim 11, wherein the natural waxes are chosen from microcrystalline waxes; paraffin waxes; petrolatum waxes; paraffin waxes; ozokerites; montan waxes; beeswaxes; lanolin and derivatives thereof; candelilla waxes; ouricury waxes; carnauba waxes; Japan waxes; cocoa butter, cork fibre and sugarcane waxes; hydrogenated oils which are solid at 25° C.; and fatty esters and glycerides which are solid at 25° C.

13. The composition according to claim 11, wherein the synthetic waxes are chosen from silicone waxes, waxes obtained by Fischer-Tropsch synthesis, polyethylene waxes, polytetrafluoroethylene (PTFE) waxes, and mixtures thereof.

14. The composition according to claim 1, where the wax particles comprise a mixture of at least one natural wax and at least one synthetic wax.

15. The composition according to claim 1, wherein the wax particles comprise a mixture of at least two natural waxes and/or a mixture of at least two synthetic waxes.

16. The composition according to claim 1, wherein the wax particles are carnauba wax particles with a melting point ranging from 83° C. to 86° C., a density of 0.99 g/cm³, and a mean particle size ranging from 6 μm to 8 μm.

17. The composition according to claim 1, wherein the wax particles comprise a mixture of carnauba wax and polyethylene wax and have a melting point ranging from 107° C. to 114° C., a density ranging from 0.96 g/cm³ to 0.97g/cm³, and a mean particle size ranging from 4 μm to 6 μm.

18. The composition according to claim 1, wherein the wax particles comprise polytetrafluoroethylene and have a melting point of greater than 316° C., a density of approximately 2.2, and a mean particle size ranging from 5 μm to 13 μm.

19. The composition according to claim 1, wherein the wax particles comprise a mixture of polyethylene wax and polytetrafluoroethylene wax and have a melting point ranging from 115° C. to 125° C., a density ranging from 0.99 g/cm³ to 1.1 g/cm³, and a mean particle size ranging from 9 μm to 12 μm.

20. The composition according to claim 1, wherein the wax particles comprise polyethylene and have a melting point of greater than 100° C., a density ranging from 0.96 g/cm³ to 0.98 g/cm³, and a mean particle size ranging from 5 μm to 40 μm.

21. The composition according to claim 20, wherein the polyethylene particles have a mean size ranging from 1 μm to 12 μm, a melting point ranging from 105° C. to 135° C., and a density ranging from 0.94 g/cm³ to 0.97g/cm³.

22. The composition according to claim 1, wherein the plurality of wax particles are present in an amount ranging from 0.5% to 15% by weight, with respect to the total weight of the composition.

23. The composition according to claim 22, wherein the plurality of wax particles are present in an amount ranging from 1% to 10% by weight, with respect to the total weight of the composition.

24. The composition according to claim 1, further comprising at least one additional deodorant active agent.

25. The composition according to claim 24, wherein the at least one additional deodorant active agent is chosen from bacteriostatic and bactericidal agents.

26. The composition according to claim 24, wherein the at least one additional deodorant active agent is chosen from 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl) urea (Triclocarban) and 3,7,11 -trimethyidodeca-2,5,10-trienol (Farnesol); sodium bicarbonate; quaternary ammonium salts; zinc salts; chlorhexidine and salts thereof; diglycerol monocaprate, diglycerol monolaurate and glycerol monolaurate; and polyhexamethylene biguanide salts.

27. The composition according to claim 24, wherein the at least one additional deodorant active agent is present in an amount ranging from 0.001% to 40% by weight, with respect to the total weight of the composition.

28. The composition according to claim 27, wherein the at least one additional deodorant active agent is present in an amount ranging from 0.1% to 25% by weight, with respect to the total weight of the composition.

29. The composition according to claim 1, further comprising at least one volatile or nonvolatile emollient oil of silicone or hydrocarbon nature.

30. The composition according to claim 1, further comprising at least one suspending agent.

31. The composition according to claim 1, further comprising at least one organic powder.

32. The composition according to claim 1, further comprising at least one cosmetic adjuvant chosen from waxes, softeners, antioxidants, opacifiers, stabilizers, moisturizing agents, vitamins, fragrances, bactericides, preservatives, polymers, and thickening agents.

33. The composition according to claim 1, further comprising at least one structuring or gelling agent.

34. The composition according to claim 1, wherein the composition is pressurized and packaged in an aerosol comprising at least one propellant.

35. A process for reducing the flow of sweat and/or making, improving or reducing the unpleasant odor resulting from the decomposition of human sweat by bacteria comprising applying, to-the surface of skin, an effective amount of a composition comprising, in an oil-in-water emulsion: (A) at least one antiperspirant active agent; and (B) a plurality of wax particles with a mean size of less than 50 μm and a melting point of greater than 80° C.

36. A process for treating perspiration comprising applying, to the surface of skin, an effective amount of a composition comprising, in an oil-in-water emulsion: (A) at least one antiperspirant active agent; and (B) a plurality of wax particles with a mean size of less than 50 μm and a melting point of greater than 80° C.

37. A process for treating human body odors comprising applying, to the surface of skin, an effective amount of a composition comprising, in an oil-in-water emulsion: (A) at least one antiperspirant active agent; and (B) a plurality of wax particles with a mean size of less than 50 μm and a melting point of greater than 80° C.

38. A method for reducing the drying time and the stickiness effect of an antiperspirant composition comprising adding a plurality of wax particles having a mean size of less than 50 μm and a melting point of greater than 80° C. to the antiperspirant composition.
